(19) United States
(12) Patent Application Publication
(10) Pub. No.: US 2026/0176250 A1
(43) Pub. Date: Jun. 25, 2026

(54) TRIAZINONE DERIVATIVES AS NLRP3 INHIBITORS

(71) Applicant: Hoffmann-La Roche Inc., Little Falls, NJ (US)

(72) Inventors: Lea Aurelie BOUCHE, Basel (CH); Wolfgang GUBA, Müllheim (DE); Georg JAESCHKE, Basel (CH); Stephanie Katharina MESCH, Basel (CH); Jonathan Martin SHANNON, Nottingham (GB)

(73) Assignee: Hoffmann-La Roche Inc., Little Falls, NJ (US)

(21) Appl. No.: 19/128,448

(22) PCT Filed: Nov. 7, 2023
(86) PCT No.: PCT/EP2023/080902

§ 371 Date: May 8, 2025

(30) Foreign Application Priority Data
Nov. 9, 2022 (EP) 22206287.9

(51) Int. Cl. C07D 401/12; A61K 31/53

(52) U.S. Cl. CPC C07D 401/12; A61K 31/53

(57) ABSTRACT

The invention relates to a novel compound having the formula 6-[[(3R)-1-Ethyl-3-piperidyl]amino]-3-(4-hydroxyindan-5-yl)-4-methyl-1,2,4-triazin-5-one or 6-[[(3R)-1-ethyl-3-piperidyl]amino]-3-(2-hydroxy-3-bicyclo[4.2.0]octa-1,3,5-trienyl)-4-methyl-1,2,4-triazin-5-one, and pharmaceutically acceptable salts thereof, compositions including the compound and methods of using the compound.

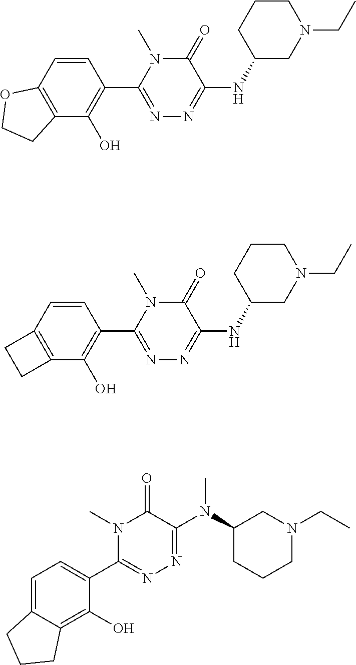 [SMILES: CCN1CCC[C@@H](Nc2nnc(-c3ccc4c(c3O)CCO4)n(C)c2=O)C1 ; CCN1CCC[C@@H](Nc2nnc(-c3ccc4c(c3O)CC4)n(C)c2=O)C1 ; CCN1CCC[C@@H](N(C)c2nnc(-c3ccc4c(c3O)CCC4)n(C)c2=O)C1]

### FIELD OF THE INVENTION

The present invention relates to an organic compound useful for therapy and/or prophylaxis in a mammal, and in particular to a compound that modulate NLRP3 inhibition.

The present invention provides a novel compound selected from
6-[(3R)-1-ethyl-3-piperidyl]amino]-3-(4-hydroxyindan-5-yl)-4-methyl-1,2,4-triazin-5-one; and
6-[[(3R)-1-ethyl-3-piperidyl]amino]-3-(2-hydroxy-3-bicyclo[4.2.0]octa-1,3,5-trienyl)-4-methyl-1,2,4-triazin-5-one;
6-[(3R)-1-Ethyl-3-piperidyl]-methyl-amino]-3-(4-hydroxyindan-5-yl)-4-methyl-1,2,4-triazin-5-one; and
or a pharmaceutically acceptable salt thereof.

### BACKGROUND OF THE INVENTION

The NOD-like receptor (NLR) family, pyrin domain-containing protein 3 (NLRP3) inflammasome is a component of the inflammatory process, and its aberrant activity is pathogenic in inherited disorders such as cryopyrin-associated periodic syndromes (CAPS) and complex diseases such as multiple sclerosis, type 2 diabetes, Alzheimer's disease and atherosclerosis.

NLRP3 is an intracellular signaling molecule that senses many pathogen-derived, environmental and host-derived factors. Upon activation, NLRP3 binds to apoptosis-associated speck-like protein containing a caspase activation and recruitment domain (ASC). ASC then polymerises to form a large aggregate known as an ASC speck. Polymerised ASC in turn interacts with the cysteine protease caspase-1 to form a complex termed the inflammasome. This results in the activation of caspase-1, which cleaves the precursor forms of the proinflammatory cytokines IL-1β and IL-18 (termed pro-IL-1β and pro-IL-18 respectively) to thereby activate these cytokines. Caspase-1 also mediates a type of inflammatory cell death known as pyroptosis. The ASC speck can also recruit and activate caspase-8, which can process pro-IL-1B and pro-IL-18 and trigger apoptotic cell death.

Caspase-1 cleaves pro-IL-1β and pro-IL-18 to their active forms, which are secreted from the cell. Active caspase-1 also cleaves gasdermin-D to trigger pyroptosis. Through its control of the pyroptotic cell death pathway, caspase-1 also mediates the release of alarmin molecules such as IL-33 and high mobility group box 1 protein (HMGB1). Caspase-1 also cleaves intracellular IL-1R2 resulting in its degradation and allowing the release of IL-1α. In human cells caspase-1 may also control the processing and secretion of IL-37. A number of other caspase-1 substrates such as components of the cytoskeleton and glycolysis pathway may contribute to caspase-1-dependent inflammation.

NLRP3-dependent ASC specks are released into the extracellular environment where they can activate caspase-1, induce processing of caspase-1 substrates and propagate inflammation.

Active cytokines derived from NLRP3 inflammasome activation are important drivers of inflammation and interact with other cytokine pathways to shape the immune response to infection and injury. For example, IL-1β signalling induces the secretion of the pro-inflammatory cytokines IL-6 and TNF. IL-1β and IL-18 synergise with IL-23 to induce IL-17 production by memory CD4 Th17 cells and by γδ T cells in the absence of T cell receptor engagement. IL-18 and IL-12 also synergise to induce IFN-γ production from memory T cells and NK cells driving a Th1 response.

The inherited CAPS diseases Muckle-Wells syndrome (MWS), familial cold autoinflammatory syndrome (FCAS) and neonatal-onset multisystem inflammatory disease (NOMID) are caused by gain-of-function mutations in NLRP3, thus defining NLRP3 as a critical component of the inflammatory process. NLRP3 has also been implicated in the pathogenesis of a number of complex diseases, notably including metabolic disorders such as type 2 diabetes, atherosclerosis, obesity and gout.

A role for NLRP3 in diseases of the central nervous system is emerging, and lung diseases have also been shown to be influenced by NLRP3. NLRP3 has also been suggested to have a role in a number of central nervous system conditions, including Parkinson's disease (PD), Alzheimer's disease (AD), dementia, Huntington's disease, cerebral malaria, brain injury from pneumococcal meningitis (Walsh et al., Nature Reviews, 15:84-97, 2014, and Dempsey et al. Brain. Behav. Immun. 201761:306-316). NLRP3 has also been shown to play a role in a number of lung diseases including chronic obstructive pulmonary disorder (COPD), asthma (including steroid-resistant asthma), asbestosis, and silicosis (De Nardo et al., Am. J. Pathol., 184:42-54, 2014 and Kim et al. Am J Respir Crit Care Med. 2017 196 (3): 283-97). Furthermore, NLRP3 has a role in the development of liver disease, kidney disease and aging. Many of these associations were defined using Nlrp3^{-/-} mice, but there have also been insights into the specific activation of NLRP3 in these diseases. In type 2 diabetes mellitus (T2D), the deposition of islet amyloid polypeptide in the pancreas activates NLRP3 and IL-1β signalling, resulting in cell death and inflammation.

Several small molecules have been shown to inhibit the NLRP3 inflammasome. Glyburide inhibits IL-1β production at micromolar concentrations in response to the activation of NLRP3 but not NLRC4 or NLRP1. Other previously characterised weak NLRP3 inhibitors include parthenolide, 3,4-methylenedioxy-β-nitrostyrene and dimethyl sulfoxide (DMSO), although these agents have limited potency and are nonspecific.

Current treatments for NLRP3-related diseases include biologic agents that target IL-1. These are the recombinant IL-1 receptor antagonist anakinra, the neutralizing IL-1β antibody canakinumab and the soluble decoy IL-1 receptor rilonacept. These approaches have proven successful in the treatment of CAPS, and these biologic agents have been used in clinical trials for other IL-1β-associated diseases.

There is a need to provide a compound with improved pharmacological and/or physiological and/or physicochemical properties and/or those that provide a useful alternative to known compounds. In particular, compounds with good activity and improved in-vitro stability.

### SUMMARY OF THE INVENTION

The present invention provides a novel compound selected from
6-[[(3R)-1-ethyl-3-piperidyl]amino]-3-(4-hydroxyindan-5-yl)-4-methyl-1,2,4-triazin-5-one; and
6-[[(3R)-1-ethyl-3-piperidyl]amino]-3-(2-hydroxy-3-bicyclo[4.2.0]octa-1,3,5-trienyl)-4-methyl-1,2,4-triazin-5-one;
6-[(3R)-1-Ethyl-3-piperidyl]-methyl-amino]-3-(4-hydroxyindan-5-yl)-4-methyl-1,2,4-triazin-5-one; and
or a pharmaceutically acceptable salt thereof.

The term “pharmaceutically acceptable salts” refers to those salts which retain the biological effectiveness and properties of the free bases or free acids, which are not biologically or otherwise undesirable. The salts are formed with inorganic acids such as trifluoroacetic acid, hydrochloric acid, hydrobromic acid, sulfuric acid, nitric acid, phosphoric acid, particularly hydrochloric acid, and organic acids such as formic acid, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, N-acetylcystein. In addition these salts may be prepared from addition of an inorganic base or an organic base to the free acid. Salts derived from an inorganic base include, but are not limited to, the sodium, potassium, lithium, ammonium, calcium, magnesium salts. Salts derived from organic bases include, but are not limited to salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, lysine, arginine, N-ethylpiperidine, piperidine, polyamine resins. The compound of the formula can also be present in the form of zwitterions. Particularly preferred pharmaceutically acceptable salts of compounds of the formula are the salts formed with formic acid and the salts formed with hydrochloric acid yielding a hydrochloride, dihydrochloride or trihydrochloride salt.

The abbreviation uM means microMolar and is equivalent to the symbol μM.

The abbreviation uL means microliter and is equivalent to the symbol μL.

The abbreviation ug means microgram and is equivalent to the symbol μg.

Also an embodiment of the present invention provides a compound according to those described herein and pharmaceutically acceptable salts or esters thereof, in particular a compound according to those as described herein and pharmaceutically acceptable salts thereof, more particularly a compound according to those as described herein.

Particular examples of compounds as described herein are selected from
6-[[(3R)-1-ethyl-3-piperidyl]amino]-3-(4-hydroxyindan-5-yl)-4-methyl-1,2,4-triazin-5-one;
6-[(3R)-1-ethyl-3-piperidyl]amino]-3-(2-hydroxy-3-bicyclo[4.2.0]octa-1,3,5-trienyl)-4-methyl-1,2,4-triazin-5-one;
and pharmaceutically acceptable salts thereof.

An other particular example of compounds as described herein is 6-[(3R)-1-Ethyl-3-piperidyl]-methyl-amino]-3-(4-hydroxyindan-5-yl)-4-methyl-1,2,4-triazin-5-one, or a pharmaceutically acceptable salt thereof.

An embodiment of the present invention provides a compound of formula 6-[(3R)-1-ethyl-3-piperidyl]amino]-3-(4-hydroxyindan-5-yl)-4-methyl-1,2,4-triazin-5-one, or a pharmaceutically acceptable salt thereof.

An embodiment of the present invention provides a compound of formula 6-[(3R)-1-ethyl-3-piperidyl]amino]-3-(2-hydroxy-3-bicyclo[4.2.0]octa-1,3,5-trienyl)-4-methyl-1,2,4-triazin-5-one, or a pharmaceutically acceptable salt thereof.

Another embodiment of the invention provides a pharmaceutical composition or medicament containing a compound of the invention and a therapeutically inert carrier, diluent or excipient, as well as a method of using the compound of the invention to prepare such composition and medicament. In one example, the compound may be formulated by mixing at ambient temperature at the appropriate pH, and at the desired degree of purity, with physiologically acceptable carriers, i.e., carriers that are non-toxic to recipients at the dosages and concentrations employed into a galenical administration form. The pH of the formulation depends mainly on the particular use and the concentration of compound, but preferably ranges anywhere from about 3 to about 8. In one example, the compound of the formula is formulated in an acetate buffer, at pH 5. In another embodiment, the compound of the formula is sterile. The compound may be stored, for example, as a solid or amorphous composition, as a lyophilized formulation or as an aqueous solution.

Compositions are formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

A compound of the invention may be administered by any suitable means, including oral, topical (including buccal and sublingual), rectal, vaginal, transdermal, parenteral, subcutaneous, intraperitoneal, intrapulmonary, intradermal, intrathecal and epidural and intranasal, and, if desired for local treatment, intralesional administration. Parenteral infusions include intramuscular, intravenous, intraarterial, intraperitoneal, or subcutaneous administration.

A compound of the present invention may be administered in any convenient administrative form, e.g., tablets, powders, capsules, solutions, dispersions, suspensions, syrups, sprays, suppositories, gels, emulsions, patches, etc. Such compositions may contain components conventional in pharmaceutical preparations, e.g., diluents, carriers, pH modifiers, sweeteners, bulking agents, and further active agents.

A typical formulation is prepared by mixing a compound of the present invention and a carrier or excipient. Suitable carriers and excipients are well known to those skilled in the art and are described in detail in, e.g., Ansel, Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; and Rowe, Raymond C. Handbook of Pharmaceutical Excipients. Chicago, Pharmaceutical Press, 2005. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide an elegant presentation of the drug (i.e., a compound of the present invention or pharmaceutical composition thereof) or aid in the manufacturing of the pharmaceutical product (i.e., medicament).

A compound and its pharmaceutically acceptable salts can be processed with pharmaceutically inert, inorganic or organic adjuvants for the production of tablets, coated tablets, dragées, hard gelatin capsules, injection solutions or topical formulations Lactose, corn starch or derivatives thereof, talc, stearic acid or its salts etc. can be used, for example, as such adjuvants for tablets, dragées and hard gelatin capsules.

Suitable adjuvants for soft gelatin capsules, are, for example, vegetable oils, waxes, fats, semi-solid substances and liquid polyols, etc.

Suitable adjuvants for the production of solutions and syrups are, for example, water, polyols, saccharose, invert sugar, glucose, etc.

Suitable adjuvants for injection solutions are, for example, water, alcohols, polyols, glycerol, vegetable oils, etc.

Suitable adjuvants for suppositories are, for example, natural or hardened oils, waxes, fats, semi-solid or liquid polyols, etc.

Suitable adjuvants for topical ocular formulations are, for example, cyclodextrins, mannitol or many other carriers and excipients known in the art.

Moreover, the pharmaceutical preparations can contain preservatives, solubilizers, viscosity-increasing substances, stabilizers, wetting agents, emulsifiers, sweeteners, colorants, flavorants, salts for varying the osmotic pressure, buffers, masking agents or antioxidants. They can also contain still other therapeutically valuable substances.

The dosage can vary in wide limits and will, of course, be fitted to the individual requirements in each particular case. In general, in the case of oral administration a daily dosage of about 0.1 mg to 20 mg per kg body weight, preferably about 0.5 mg to 4 mg per kg body weight (e.g. about 300 mg per person), divided into preferably 1-3 individual doses, which can consist, for example, of the same amounts, should it be appropriate. In the case of topical administration, the formulation can contain 0.001% to 15% by weight of medicament and the required dose, which can be between 0.1 and 25 mg in can be administered either by single dose per day or per week, or by multiple doses (2 to 4) per day, or by multiple doses per week It will, however, be clear that the upper or lower limit given herein can be exceeded when this is shown to be indicated.

An embodiment of the present invention is a compound according to the invention as described herein for use as a therapeutically active substance.

An embodiment of the present invention is a compound according to the invention as described herein for use in the treatment or prevention of a disease, disorder or condition, wherein the disease, disorder or condition is responsive to NLRP3 inhibition.

An embodiment of the present invention is a compound according to the invention as described herein for the treatment or prophylaxis of a disease, disorder or condition, wherein the disorder or condition is responsive to NLRP3 inhibition.

As used herein, the term “NLRP3 inhibition” refers to the complete or partial reduction in the level of activity of NLRP3 and includes, for example, the inhibition of active NLRP3 and/or the inhibition of activation of NLRP3.

There is evidence for a role of NLRP3-induced IL-1 and IL-18 in the inflammatory responses occurring in connection with, or as a result of, a multitude of different disorders (Menu et al., Clinical and Experimental Immunology, 166:1-15, 2011; Strowig et al., Nature, 481:278-286, 2012).

In one embodiment, the disease, disorder or condition is selected from:
(i) inflammation;
(ii) an auto-immune disease;
(iii) cancer;
(iv) an infection;
(v) a central nervous system disease;
(vi) a metabolic disease;
(vii) a cardiovascular disease;
(viii) a respiratory disease;
(ix) a liver disease;
(x) a renal disease;
(xi) an ocular disease;
(xii) a skin disease;
(xiii) a lymphatic condition;
(xiv) a psychological disorder;
(xv) graft versus host disease;
(xvi) allodynia;
(xvii) a condition associated with diabetes; and
(xviii) any disease where an individual has been determined to carry a germline or somatic non-silent mutation in NLRP3

In another embodiment, the disease, disorder or condition is selected from:
(i) cancer;
(ii) an infection;
(iii) a central nervous system disease;
(iv) a cardiovascular disease;
(v) a liver disease;
(vi) an ocular disease; or
(vii) a skin disease.

In a further typical embodiment of the invention, the disease, disorder or condition is inflammation. Examples of inflammation that may be treated or prevented include inflammatory responses occurring in connection with, or as a result of:
(i) a skin condition such as contact hypersensitivity, bullous pemphigoid, sunburn, psoriasis, atopical dermatitis, contact dermatitis, allergic contact dermatitis, seborrhoetic dermatitis, lichen planus, scleroderma, pemphigus, epidermolysis bullosa, urticaria, erythemas, or alopecia;
(ii) a joint condition such as osteoarthritis, systemic juvenile idiopathic arthritis, adult-onset Still's disease, relapsing polychondritis, rheumatoid arthritis, juvenile chronic arthritis, gout, or a seronegative spondyloarthropathy (e.g. ankylosing spondylitis, psoriatic arthritis or Reiter's disease);
(iii) a muscular condition such as polymyositis or myasthenia gravis;
(iv) a gastrointestinal tract condition such as inflammatory bowel disease (including Crohn's disease and ulcerative colitis), colitis, gastric ulcer, Coeliac disease, proctitis, pancreatitis, eosinopilic gastro-enteritis, mastocytosis, antiphospholipid syndrome, or a food-related allergy which may have effects remote from the gut (e.g., migraine, rhinitis or eczema);
(v) a respiratory system condition such as chronic obstructive pulmonary disease (COPD), asthma (including eosinophilic, bronchial, allergic, intrinsic, extrinsic or dust asthma, and particularly chronic or inveterate asthma, such as late asthma and airways hyper-responsiveness), bronchitis, rhinitis (including acute rhinitis, allergic rhinitis, atrophic rhinitis, chronic rhinitis, rhinitis caseosa, hypertrophic rhinitis, rhinitis pumlenta, rhinitis sicca, rhinitis medicamentosa, membranous rhinitis, seasonal rhinitis e.g. hay fever, and vasomotor rhinitis), sinusitis, idiopathic pulmonary fibrosis (IPF), sarcoidosis, farmer's lung, silicosis, asbestosis, volcanic ash induced inflammation, adult respiratory distress syndrome, hypersensitivity pneumonitis, or idiopathic interstitial pneumonia;
(vi) a vascular condition such as atherosclerosis, Behcet's disease, vasculitides, or Wegener's granulomatosis;
(vii) an autoimmune condition such as systemic lupus erythematosus, Sjögren's syndrome, systemic sclerosis, Hashimoto's thyroiditis, type I diabetes, idiopathic thrombocytopenia purpura, or Graves disease;
(viii) an ocular condition such as uveitis, allergic conjunctivitis, or vernal conjunctivitis;
(ix) a nervous condition such as multiple sclerosis or encephalomyelitis;
(x) an infection or infection-related condition, such as Acquired Immunodeficiency Syndrome (AIDS), acute or chronic bacterial infection, acute or chronic parasitic infection, acute or chronic viral infection, acute or chronic fungal infection, meningitis, hepatitis (A, B or C, or other viral hepatitis), peritonitis, pneumonia, epiglottitis, malaria, dengue hemorrhagic fever, leishmaniasis, streptococcal myositis, Mycobacterium tuberculosis (including Mycobacterium tuberculosis and HIV co-infection), Mycobacterium avium intracellulare, Pneumocystis carinii pneumonia, orchitis/epidydimitis, legionella, Lyme disease, influenza A, Epstein-Barr virus infection, viral encephalitis/aseptic meningitis, or pelvic inflammatory disease;
(xi) a renal condition such as mesangial proliferative glomerulonephritis, nephrotic syndrome, nephritis, glomerular nephritis, obesity related glomerulopathy, acute renal failure, acute kidney injury, uremia, nephritic syndrome, kidney fibrosis including chronic crystal nephropathy, or renal hypertension;
(xii) a lymphatic condition such as Castleman's disease;
(xiii) a condition of, or involving, the immune system, such as hyper IgE syndrome, lepromatous leprosy, familial hemophagocytic lymphohistiocytosis, or graft versus host disease;
(xiv) a hepatic condition such as chronic active hepatitis, non-alcoholic steatohepatitis (NASH), alcohol-induced hepatitis, non-alcoholic fatty liver disease (NAFLD), alcoholic fatty liver disease (AFLD), alcoholic steatohepatitis (ASH), primary biliary cirrhosis, fulminant hepatitis, liver fibrosis, or liver failure;
(xv) a cancer, including those cancers listed above;
(xvi) a burn, wound, trauma, haemorrhage or stroke;
(xvii) radiation exposure;
(xviii) a metabolic disease such as type 2 diabetes (T2D), atherosclerosis, obesity, gout or pseudo-gout; and/or
(xix) pain such as inflammatory hyperalgesia, pelvic pain, allodynia, neuropathic pain, or cancer-induced bone pain.

An embodiment of the present invention is a compound according to the invention as described herein for the treatment or prophylaxis of a disease, disorder or condition selected from:
(i) inflammation;
(ii) an auto-immune disease;
(iii) cancer;
(iv) an infection;
(v) a central nervous system disease;
(vi) a metabolic disease;
(vii) a cardiovascular disease;
(viii) a respiratory disease;
(ix) a liver disease;
(x) a renal disease;
(xi) an ocular disease;
(xii) a skin disease;
(xiii) a lymphatic condition;
(xiv) a psychological disorder;
(XV) graft versus host disease;
(xvi) allodynia;
(xvii) a condition associated with diabetes; and
(xviii) any disease where an individual has been determined to carry a germline or somatic non-silent mutation in NLRP3.

An embodiment of the present invention is the use of a compound according to the invention as described herein in the treatment or prophylaxis of a disease, disorder or condition, wherein the disease, disorder or condition is responsive to NLRP3 inhibition.

An embodiment of the present invention is the use of a compound according to the invention as described herein in the treatment or prophylaxis of a disease, disorder or condition selected from Alzheimer's disease and Parkinson's disease.

An embodiment of the present invention is the use a compound according to the invention as described herein for use in the treatment or prophylaxis of a disease, disorder or condition selected from Asthma or COPD.

An embodiment of the present invention is a compound according to the invention as described herein for the treatment or prophylaxis of a disease, disorder or condition selected from Alzheimer's disease and Parkinson's disease.

An embodiment of the present invention is a compound according to the invention as described herein for the treatment or prophylaxis of a disease, disorder or condition selected from Asthma or COPD.

An embodiment of the present invention is the use of a compound according to the invention as described herein for preparation of a medicament for the treatment or prophylaxis of a disease, disorder or condition selected from Alzheimer's disease and Parkinson's disease.

An embodiment of the present invention is the use of a compound according to the invention as described herein for the preparation of a medicament for the treatment or prophylaxis of a disease, disorder or condition selected from Asthma or COPD.

An embodiment of the present invention is a method of treatment or prophylaxis of a disease, disorder or condition selected from Alzheimer's disease and Parkinson's disease, which method comprises administering an effective amount of a compound according to the invention as described herein.

An embodiment of the present invention is a method of treatment or prophylaxis of a disease, disorder or condition selected from Asthma or COPD, which method comprises administering an effective amount of a compound according to the invention as described herein.

An embodiment of the present invention relates to a method of inhibiting NLRP3, which method comprises administering an effective amount of a compound according to the invention as described herein.

Also an embodiment of the present invention are compounds of the formula as described herein, when manufactured according to any one of the described processes.

An embodiment of the present invention is a pharmaceutical composition comprising a compound according to the invention as described herein and a therapeutically inert carrier.

### Assay Procedures

### NLRP3 and Pyroptosis

It is well established that the activation of NLRP3 leads to cell pyroptosis and this feature plays an important part in the manifestation of clinical disease (Yan-gang Liu et al., Cell Death & Disease, 2017, 8 (2), e2579; Alexander Wree et al., Hepatology, 2014, 59 (3), 898-910; Alex Baldwin et al., Journal of Medicinal Chemistry, 2016, 59 (5), 1691-1710; Ema Ozaki et al., Journal of Inflammation Research, 2015, 8, 15-27; Zhen Xie & Gang Zhao, Neuroimmunology Neuroinflammation, 2014, 1 (2), 60-65; Mattia Cocco et al., Journal of Medicinal Chemistry, 2014, 57 (24), 10366-10382; T. Satoh et al., Cell Death & Disease, 2013, 4, e644). Therefore, it is anticipated that inhibitors of NLRP3 will block pyroptosis, as well as the release of pro-inflammatory cytokines (e.g. IL-1β) from the cell.

### THP-1 Cells: Culture and Preparation

THP-1 cells (ATCC #TIB-202) were grown in RPMI containing L-glutamine (Gibco #11835) supplemented with 1 mM sodium pyruvate (Sigma #S8636) and penicillin (100 units/ml)/streptomycin (0.1 mg/ml) (Sigma #P4333) in 10% Fetal Bovine Serum (FBS) (Sigma #F0804). The cells were routinely passaged and grown to confluency (˜106 cells/ml). On the day of the experiment, THP-1 cells were harvested and resuspended into RPMI medium (without FBS). The cells were then counted and viability (>90%) checked by Trypan blue (Sigma #T8154). Appropriate dilutions were made to give a concentration of 625,000 cells/ml. To this diluted cell solution was added LPS (Sigma #L4524) to give a 1 μg/ml Final Assay Concentration (FAC). 40 μl of the final preparation was aliquoted into each well of a 96-well plate. The plate thus prepared was used for compound screening.

### THP-1 Cells Pyroptosis Assay

The following method step-by-step assay was followed for compound screening.
1. Seed THP-1 cells (25,000 cells/well) containing 1.0 μg/ml LPS in 40 μl of RPMI medium (without FBS) in 96-well, black walled, clear bottom cell culture plates coated with poly-D-lysine (VWR #734-0317)
2. Add 5 μl compound (8 points half-log dilution, with 10 μM top dose) or vehicle (DMSO 0.1% FAC) to the appropriate wells
3. Incubate for 3 hours at 37° C., 5% CO₂
4. Add 5 μl nigericin (Sigma #N7143) (FAC 5 μM) to all wells
5. Incubate for 1 hr at 37° C., 5% CO₂
6. At the end of the incubation period, spin plates at 300xg for 3 mins and remove supernatant
7. Then add 50 μl of resazurin (Sigma #R7017) (FAC 100 μM resazurin in RPMI medium without FBS) and incubate plates for a further 1-2 hours at 37° C. and 5% CO₂
8. Plates were read in an Envision reader at Ex 560 nm and Em 590 nm
9. IC₅₀ data is fitted to a non-linear regression equation (log inhibitor vs response-variable slope 4-parameters)

The results of the pyroptosis assay are summarised in Table 1 below as THP IC₅₀.

### Human Whole Blood IL-1β Release Assay

For systemic delivery, the ability to inhibit NLRP3 when the compounds are present within the bloodstream is of great importance. For this reason, the NLRP3 inhibitory activity of a number of compounds in human whole blood was investigated in accordance with the following protocol.

Human whole blood in Li-heparin tubes was obtained from healthy donors from a volunteer donor panel.
1. Plate out 80 μl of whole blood containing 1 μg/ml of LPS in 96-well, clear bottom cell culture plate (Corning #3585)
2. Add 10 μl compound (8 points half-log dilution with 10 μM top dose) or vehicle (DMSO 0.1% FAC) to the appropriate wells
3. Incubate for 3 hours at 37° C., 5% CO₂
4. Add 10 μl nigericin (Sigma #N7143) (10 μM FAC) to all wells
5. Incubate for 1 hr at 37° C., 5% CO₂
6. At the end of the incubation period, spin plates at 300xg for 5 mins to pellet cells and remove 20 μl of supernatant and add to 96-well v-bottom plates for IL-1β analysis (note: these plates containing the supernatants can be stored at −80° C. to be analysed at a later date)
7. IL-1β was measured according to the manufacturer protocol (Perkin Elmer-AlphaLisa IL-1 Kit AL220F-5000)
8. IC₅₀ data is fitted to a non-linear regression equation (log inhibitor vs response-variable slope 4-parameters)

The results of the human whole blood assay are summarised in Table 1 below as HWB IC₅₀.

hERG Screening Assay

In the drug development process of small molecules, one of the most frequent adverse side effects, leading to the failure of drugs, is the cardiac arrhythmias. Such failure is often related to the capacity of the drug to inhibit the human ether-à-go-go-related gene (hERG) cardiac potassium channel. Having no or low inhibition of the hERG cardiac potassium channel is therefore considered as beneficial.

### Cells

The CHO crelox hERG cell line (ATCC reference Nr. PTA-6812, female Chinese hamster cells) was generated and validated at Roche. Ready-to-use frozen instant CHO-hERG cells were cryopreserved at Evotec (Germany) and used directly in the experiments.

### Experimental Solutions

The extracellular solution contains (in mM): NaCl 150; KCl 4; CaCl₂) 1; MgCl₂ 1; HEPES 10; pH 7.2-7.4 with NaOH, osmolarity 290-330 mOsm. The internal solution contains (in mM): KCl, 10; KF, 100; NaCl, 10; HEPES, 10; EGTA, 20; pH=7.0-7.4 with KOH, osmolarity 260-300 mOsm.

### Electrophysiology

The effects of a compound on hERG K+-currents parameters will be evaluated at 2 concentrations in at least 4 cells.

The hERG test is performed using automated patch clamp system SynchroPatch® 384 (Nanion Technologies GmbH, Germany). K+ currents are measured with the patch-voltage-clamp technique in the whole-cell configuration at 35-37° C.

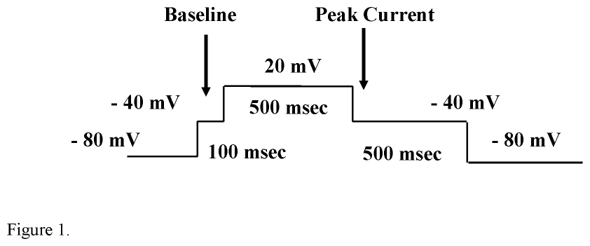

Cells were held at a resting voltage of −80 mV and they were stimulated by a voltage pattern shown in FIG. 1 (pulse pattern used to elicit outward K⁺ current at 35-37° C.) to activate hERG channels and conduct outward IKhERG current, at a stimulation frequency of 0.1 Hz (6 bpm)

### Data Analysis

The amplitudes of IKhERG were recorded in each concentration of drug and they were compared to the vehicle control values (taken as 100%) to define fractional blocks. The concentration-response data were fitted with the following relationship:

$$I ( C ) = \frac{100}{1 + {( C / IC 50 )}^{h}}$$

$$where C is the concentration ,$$

$${IC}_{50} is the concentration producting 50 \% block$$

$$h is the Hill coefficient .$$

Concentration-response curves were fitted by non-linear regression analysis using EworkBook suite (ID Business Solutions Ltd, UK). Data fit was done with the 4 Parameter Logistic Model (fit=(A+ (B/(1+ ((x/C)\hat{ }D)))), where A=0 and B=100). The results of the hERG assay are summarised in Table 2 below as hERG IC₂₀.

### Transcellular P-Gp Assay:

The general assay uses transfected LLC-PK1 cells (porcine kidney epithelial cells) over-expressing human or mouse P-gp, cultured on 96 well semi-permeable filter membrane plates, where they form a polarized monolayer with tight junctions, and act as a barrier between the apical and basolateral compartment.

P-gp is expressed in the apical-facing membrane of the monolayer.

The tightness of the cell monolayer and functional activity of P-gp are confirmed by addition of a cell-impermeable marker, Lucifer yellow, and a reference P-gp substrate, edoxaban, respectively.

### PAMPA:

PAMPA (Parallel Artificial Membrane Permeability Assay) is a first line permeability screen for drug candidates. The PAMPA assay mimics the transcellular absorption conditions using an artificial phospholipid membrane. This assay determines a permeability value that can be used for compound optimization and ranking purposes as well as input parameters for in silico models to predict intestinal absorption.

The donor concentration is measured at t-start (reference) and compared with the donor and acceptor concentration after a certain time (t-end) to calculate the extent of passage of the compound through the membrane.

### Microsomal Stability:

Incubations of test compounds at 1 μM in microsomes (0.5 mg/mL) plus cofactor NADPH are performed in 96 well plates at 37° C. on a TECAN (Tecan Group Ltd, Switzerland) automated liquid handling system. After a 10 minutes pre-incubation step of the test compound with the microsomes, the enzymatic reaction is started by the addition of cofactors. At 1, 3, 6, 9, 15, 25, 35 and 45 minutes, aliquots of the incubations are removed and quenched with 1:3 (v/v) acetonitrile containing internal standard. Samples are then cooled and centrifuged before analysis of the supernatant by LC-MS/MS 2.

### Metabolic Stability in Hepatocytes:

### Assay Descriptions:

Biological materials. Cryopreserved hepatocytes [mouse, rat, rabbit, monkey and human (male and female; mixed)] are obtained. Viability of hepatocytes after reconstitution is at least 80% throughout the study. Ready-to-use rat/human HepatoPac® cultures [long-term hepatocyte co-cultures; pooled (n=5 for male and n=5 for female for human)] with stromal mouse fibroblasts (negative control; pooled) with the plates for incubations, application medium and maintenance medium are acquired.

Metabolism by suspended hepatocytes. Primary pooled cryopreserved hepatocytes are reconstituted in pre-warmed William's E media containing 10% FCS, 0.05 mg/mL streptomycin and 50 U/mL penicillin and 0.4 mM L-glutamine; and 0.01 mg/mL gentamicin, 0.048 mg/mL hydrocortisone and 0.004 mg/mL insulin, to a final suspension density of 1×106 cells/mL. The incubation was performed fully automatically with Liquid Handling System (Tecan) equipped with a CO₂ incubator with an orbital shaker. After the addition of a test compound at e.g. 1 μM to the wells (1×105 cells/well), the 96-well hepatocyte suspension culture plates are incubated in a 5% CO₂ at 37° C. Samples are quenched by addition of acetonitrile (including an internal standard) to the incubation well at the designated time points up to 2 h.

Metabolism by HepatoPac®. Incubations for a test article (at e.g. 1 μM, 0.1% v/v DMSO) as conducted in suspension assays are performed in 96-well plates containing either a co-culture of adherent hepatocytes with mouse fibroblast control cells or control cells alone (5% CO₂ atmosphere and 37° C.). The incubation media in human HepatoPac® is identical with that in suspended hepatocytes. At defined time points (2, 18, 26, 48, 72 and 96 h), whole wells are quenched with ice-cold acetonitrile containing an internal standard.

Samples are then centrifuged appropriately and the supernatant analyzed by LC-MS/MS. The incubation is conducted in n=1 or 2.

### Pharmacokinetics Profile of Test Substances in Minipigs:

The pharmacokinetics of the test substance was determined in minipigs following intravenous and oral administration. The experimental design consisted of three male minipigs, of which each animal received a single intravenous bolus dose, and a single oral doses with the test item. Intravenous doses were administered at a nominal dose volume of 1 mL/kg. Oral doses were administered by gavage at a nominal dose volume of 5 mL/kg. There was a washout period of at least 7 days between last sampling occasion and the next dosing occasion to the same animal. The content of all formulations was within the desired range of 85 to 115% of the nominal content. Following dosing, samples of blood (1 mL) were withdrawn from the saphenous (via cannula) or jugular vein of each animal at pre-dose, 5, 15, 30 min, 1, 2, 4, 8, 24 hours post-dose after IV dosing and at pre-dose, 15, 30 min, 1, 2, 4, 6, 8, 24, 48 hours post-dose after oral dosing. At all time points, the haematocrit was determined. The blood: plasma partitioning factor was determined at the 2 and 4 hour time point, and urine was collected as a single sample for 24 hours after dose administration. Blood samples (nominally 1 mL) were withdrawn from the saphenous (via cannula) or jugular vein of each animal into polypropylene tubes containing K2EDTAanticoagulant and was centrifuged (1500 g, 10 min, 4° C.) to prepare plasma for analysis. Residual blood cells were discarded. Plasma vials were capped and stored on wet-ice for no longer than 60 minutes before being transferred to <−50° C. storage (nominally −80° C.) prior to analysis with a specific LC-MS method.

### Toxicity Assessment of Test Substance in Minipig

The maximum tolerated dose (MTD) of the test item is determined following once daily oral (gavage) administration to the minipig. The toxicity of repeated daily administration for 14 days is then assessed. In addition, the toxicokinetic profile of the test item is characterized. Sufficient purpose-bred Göttingen minipigs are obtained from Ellegaard Göttingen, Dalmose, Denmark (Animals: 2 to 3 month age range and in a 4 to 6 kg weight range). At start of dosing animals are 4 to 5 months old and in a 6 to 9.5 kg weight range. A dose volume of 10 mL/kg is used. Individual dose volumes are calculated from the most recent body weights for each animal to target dose levels of 30, 100 and 300 mg/kg/day or others depending non MTD results. Blood samples are taken on day 1 and day 14 for the determination of drug concentration in plasma and derived toxicokinetic parameters. Animals are not fed on the day of scheduled necropsy. Each animal is anaesthetized via intramuscular injection of a Zoletil mix then killed by exsanguination. All tissues are preserved in the appropriate fixative/s. Further analysis includes food consumption, body weight, clinical pathology, and full histopathological examination of target organs.

TABLE 1
NLRP3 inhibitory activity

| | THP-1 pyroptosis | Human whole blood IL-1ß |
|---|---|---|
| | Assay | Assay IC₅₀ |
| Example No. | IC₅₀ (nM) | (nM) |
| 1 | 1.5 | 8 |
| 2 | 5.8 | 10 |
| 3 | 13 | 26 |

TABLE 2
hERG inhibition assay

| | hERG assay |
|---|---|
| Example No. | IC₂₀ (μM) |
| 1 | >10 |

TABLE 3
Metabolic Stability in Human Hepatocytes

| | Clint at 1 μM |
|---|---|
| Example No. | [μl/min/10E6 cells] |
| 1 | <0.2 |

The invention will now be illustrated by the following examples which have no limiting character.

In case the preparative examples are obtained as a mixture of enantiomers or diastereoisomers, the pure enantiomers or diastereomers can be obtained by methods described herein or by methods known to those skilled in the art, such as e.g. chiral chromatography or crystallization.

### Experimental Methods

### Abbreviations:

| | |
|---|---|
| DCM | dichloromethame |
| DMF | N,N-dimethylformamide |
| DMSO | dimethyl sulfoxide |
| EtOAc | ethyl acetate |
| Et₂O | diethyl ether |
| h, hrs | hour, hours |
| MeCN | acetonitrile |
| MeOH | methanol |
| min(s) | minute(s) |
| NMP | N-methylpyrrolidone |
| NMR | nuclear magnetic resonance spectroscopy |

### EXAMPLES

All examples and intermediates were prepared under nitrogen atmosphere if not specified otherwise.

### Intermediates

### Intermediate 1:3-Chloro-6-[ ](3R)-1-ethyl-3-piperidyl]amino]-4-methyl-1,2,4-triazin-5-one

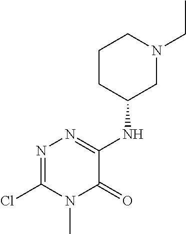 [SMILES: CCN1CCC[C@@H](Nc2nnc(Cl)n(C)c2=O)C1]

### Step A: 6-Bromo-2-[(4-methoxyphenyl)methyl]-4-methyl-1,2,4-triazine-3,5-dione

6-Bromo-4-methyl-2H-1,2,4-triazine-3,5-dione (CAS #15870-75-4, 13.8 g, 63.1 mmol, 1.0 eq) and potassium carbonate (4.84 g, 31.5 mmol, 0.50 eq) were suspended in dry DMF (125 mL) and 4-methoxybenzylchloride (10.3 mL, 75.7 mmol, 1.2 eq) was added. The reaction mixture was stirred at room temperature for 24 h. The reaction mixture was diluted with EtOAc (50 mL) and washed with 10 wt % aqueous LiCl (2×30 mL), dried using a phase separator and concentrated in vacuo. The resulting residue was purified by chromatography on silica gel (0-50% EtOAc/isohexane) to afford the title compound (15.9 g, 77% yield) as a white solid. 1H NMR (500 MHZ, DMSO-d₆) [ppm]: δ 7.33-7.25 (m, 2H), 6.97-6.89 (m, 2H), 5.00 (s, 2H), 3.74 (s, 3H), 3.20 (s, 3H).

### Step B: 6-[(3R)-1-Ethyl-3-piperidyl]amino]-2-[(4-methoxyphenyl)methyl]-4-methyl-1,2,4-triazine-3,5-dione

(3R)-1-Ethylpiperidin-3-amine (6.0 g, 46.9 mmol, 1.53 eq) and aforementioned 6-bromo-2-[(4-methoxyphenyl)methyl]-4-methyl-1,2,4-triazine-3,5-dione (10.0 g, 30.7 mmol, 1.0 eq) and cesium carbonate (20 g, 61.3 mmol, 2.0 eq) were dissolved in DMSO (125 mL) and the mixture degassed (N₂) for 5 min. The reaction vessel was evacuated and back-filled with N₂ (3×), then (rac)-BINAP Pd G3 (1 g, 1.01 mmol, 0.030 eq) was added and the reaction mixture placed under N₂, then stirred at 95° C. for 24 h. The reaction mixture was partitioned between EtOAc (500 mL) and water (500 mL). The organic phase was isolated, washed with brine (3×300 mL), dried using a phase separator and concentrated in vacuo. The resulting residue was purified by chromatography on silica gel (0-7% (0.7 N ammonia in MeOH) in DCM) to afford the title compound (10.4 g, 86% yield) as an orange oil. LCMS m/z 374.2 [M+H]⁺, ESI pos.

### Step C: 6-[(3R)-1-Ethyl-3-piperidyl]amino]-4-methyl-2H-1,2,4-triazine-3,5-dione; trifluoromethanesulfonic acid salt

Aforementioned 6-[[(3R)-1-ethyl-3-piperidyl]amino]-2-[(4-methoxyphenyl)methyl]-4-methyl-1,2,4-triazine-3,5-dione (10.4 g, 25.1 mmol, 1.0 eq) was dissolved in DCM (75 mL). Trifluoromethanesulfonic acid (3.33 mL, 37.7 mmol, 1.5 eq) was added to the reaction. The resulting solution was stirred at room temperature for 24 h. A further portion of trifluoromethanesulfonic acid (3.33 mL, 37.7 mmol, 1.5 eq) was added and the reaction mixture stirred for a further 3 h. The reaction mixture was concentrated in vacuo and the resulting residue was purified by chromatography on silica gel (0-10% (0.7 N ammonia in MeOH/DCM) to afford the title compound (17.04 g, 84% yield) as a yellow oil. LCMS m/z 254.5 [M+H]⁺, ESI pos.

### Step D: 3-Chloro-6-[[(3R)-1-ethyl-3-piperidyl]amino]-4-methyl-1,2,4-triazin-5-one

Aforementioned 6-[[(3R)-1-ethyl-3-piperidyl]amino]-4-methyl-2H-1,2,4-triazine-3,5-dione; trifluoromethanesulfonic acid salt (17.04 g, 21.1 mmol, 1.0 eq) was dissolved in phosphorus oxychloride (75.0 mL, 804.6 mmol, 38.1 eq). The reaction was stirred at 120° C. for 72 h. A 15 mL aliquot of the reaction mixture was concentrated in vacuo and the resulting residue diluted with EtOAc (200 mL), washed with 1:1 brine: saturated aqueous NaHCO₃ solution (200 mL). The organic phase was isolated and the aqueous back-extracted with EtOAc (200 mL). The combined organic extracts were dried (MgSO₄) and concentrated in vacuo to afford the title compound (1.03 g, 17% yield) as a brown oil. The remaining reaction mixture was subjected to the same workup conditions, proportionately scaled, and afforded the title compound (5.06 g, 79% yield) as a brown oil. LCMS m/z 274.4 ([37Cl]M+H)⁺, ESI pos.

### Intermediate 2:5-(4,4,5,5-Tetramethyl-1,3,2-dioxaborolan-2-yl) indan-4-ol

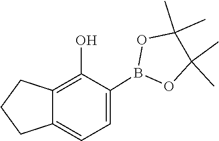 [SMILES: CC1(C)OB(c2ccc3c(c2O)CCC3)OC1(C)C]

A mixture of commercially available 5-bromoindan-4-ol (CAS #575504-23-3) (950 mg, 4.46 mmol, 1.0 eq), bis(pinacolato)diboron (5.66 g, 22.3 mmol, 5.0 eq), Pd (dppf) Cl₂·DCM complex (364.1 mg, 0.45 mmol, 0.1 eq) and potassium acetate (1.31 g, 13.4 mmol, 3.0 eq) in MeCN (40 mL) was degassed with nitrogen for 5 min after which the reaction mixture was heated to 80° C. and stirred at this temperature for 16 hours. The reaction mixture was cooled and concentrated in vacuo. The residue was purified by chromatography on silica gel (0-100% EtOAc/isohexane) to afford the title compound (211.8 mg, 17%) as a colourless oil. LCMS: no ionisation. 1H NMR (500 MHz, CDCl₃) [ppm]: δ 7.91 (s, 1H), 7.43 (d, 1H), 6.81 (d, 1H), 2.94-2.86 (m, 4H), 2.07 (d, 2H), 1.35 (s, 12H).

### Intermediate 3:3-Bromobicyclo[4.2.0]octa-1 (6),2,4-trien-2-ol

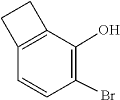

### Step A: 5-Benzyloxybicyclo[4.2.0]octa-1 (6),2,4-trien-7-ol

2.5 M Butyllithium solution in hexane (38.0 mL, 95.01 mmol, 5.0 eq) was added dropwise to stirred THF (100 mL) at 0° C. under N₂. The reaction was allowed to return to rt, then was stirred for ˜16 h. In a separate flask, 2.5 M butyllithium solution in hexane (15.2 mL, 38.0 mmol, 2.0 eq) was added dropwise to a solution of 2,2,6,6-tetramethylpiperidine (6.41 mL, 38.0 mmol, 2.0 eq) in THF (60 mL) at 0° C. and the reaction mixture was stirred for 30 mins. The first flask was then cooled to −78° C. and 3-benzyloxybromobenzene (5000 mg, 19.0 mmol, 1.0 eq; CAS #53087-13-1) in THF (25 mL) was added. The lithium 2,2,6,6-tetramethylpiperidine solution was then added dropwise, via cannula, to the reaction mixture and the reaction was stirred for 1 h. The reaction was quenched by addition of NH₄Cl (200 mL, sat aq) and was allowed to return to rt. The reaction was diluted with water (100 mL) and extracted with EtOAc (3×150 mL). The combined organic layers were dried with MgSO₄, concentrated in vacuo and purified by column chromatography on silica gel (0-10% EtOAc/isohexane) to give the title compound (3.22 g, 74% yield) as a white solid. LCMS: m/z=225.3 [M−H]⁻, ESI neg.

### Step B: 2-Benzyloxy-8-bromo-bicyclo[4.2.0]octa-1 (6),2,4-triene

Carbon tetrabromide (1.76 g, 5.3 mmol, 1.2 eq) and triphenylphosphine (2.09 g, 7.96 mmol, 1.8 eq) were added to a stirred solution of aforementioned 5-benzyloxybicyclo[4.2.0]octa-1 (6),2,4-trien-7-ol (1.0 g, 4.42 mmol, 1.0 eq) in Et20 (40 mL) at rt and the reaction was stirred for 2 days. The solution was filtered, the solid was washed with ether (40 mL) and the filtrate was concentrated in vacuo. The crude material was purified by column chromatography on silica gel (0-20% EtOAc/isohexane) to give the title compound (1186 mg, 4.1 mmol, 89% yield) as a colourless oil. LCMS: no m/z observed. 1H NMR (500 MHZ, DMSO-d₆) [ppm]: δ 7.48-7.43 (m, 2H), 7.43-7.38 (m, 2H), 7.37-7.28 (m, 2H), 6.87 (d, 1H), 6.77 (d, 1H), 5.74 (dd, 1H), 5.32 (d, 1H), 5.27 (d, 1H), 3.94-3.81 (m, 1H), 3.40-3.34 (m, 1H).

### Step C: Bicyclo[4.2.0]octa-1 (6),2,4-trien-2-ol

A mixture of aforementioned 2-benzyloxy-8-bromo-bicyclo[4.2.0]octa-1 (6),2,4-triene (1180.0 mg, 4.08 mmol, 1.0 eq) and 5% wt palladium on carbon, type R434, 50% wt water (173.7 mg, 0.04 mmol, 0.01 eq) in ethanol (25 mL) was stirred under H₂ (5 bar) for 3 h. The reaction was filtered and diluted with DCM (200 mL), then was washed with sodium thiosulfate (1×50 mL, 10% aq), sodium bicarbonate (1×50 mL, sat aq) and brine (1×50 mL). The organic phase was dried with MgSO₄, concentrated in vacuo and purified by column chromatography on silica gel (0-10% EtOAc/isohexane) to give the title compound (471.7 mg, 3.93 mmol, 91% yield) as an off-white crystalline solid. LCMS: m/z=121.1 [M+H]⁺, ESI pos.

### Step D: 3-Bromobicyclo[4.2.0]octa-1 (6),2,4-trien-2-ol

N-Bromosuccinimide (628.34 mg, 3.53 mmol, 0.95 eq) in DCM (10 mL) was added in small portions to a stirred solution of aforementioned bicyclo[4.2.0]octa-1 (6),2,4-trien-2-ol (470.0 mg, 3.72 mmol, 1.0 eq) and diisopropylamine (52.08 μL, 0.37 mmol, 0.1 eq) in DCM (40 mL) at 0° C. and the reaction was stirred for 1 h. The reaction was concentrated in vacuo and purified by column chromatography on silica gel (0-10% EtOAc/isohexane) to give the title compound (522.4 mg, 2.62 mmol, 67% yield) as a white solid. LCMS: no m/z observed. 1H NMR (500 MHZ, CDCl₃) [ppm]: δ 7.32 (d, 1H), 6.55 (d, 1H), 5.43 (s, 1H), 3.20-3.13 (m, 2H), 3.13-3.06 (m, 2H).

### EXAMPLES

### Example 1:6-[[(3R)-1-Ethyl-3-piperidyl]amino]-3-(4-hydroxyindan-5-yl)-4-methyl-1,2,4-triazin-5-one

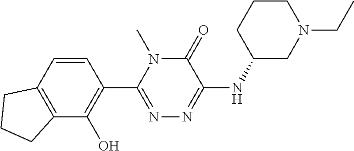 [SMILES: CCN1CCC[C@@H](Nc2nnc(-c3ccc4c(c3O)CCC4)n(C)c2=O)C1]

A mixture of aforementioned 5-(4,4,5,5-tetramethyl-1,3,2-dioxaborolan-2-yl) indan-4-Intermediate 2 (60.0 mg, 0.23 mmol, 1.25 eq), 3-chloro-6-[(3R)-1-ethyl-3-piperidyl]amino]-4-methyl-1,2,4-triazin-5-one Intermediate 1 (50.0 mg, 0.18 mmol, 1.0 eq), meCgPPh Pd G3 (CAS #. 2230788-58-4) (12.2 mg, 0.02 mmol, 0.1 eq) and potassium carbonate (76.29 mg, 0.55 mmol, 3.0 eq) in 1,4-dioxane (2 mL) and water (0.5 mL) was degassed for 5 mins with nitrogen, then the reaction was heated to 90° C. for 2 h. The reaction mixture was cooled and concentrated in vacuo. The residue was purified by chromatography on silica gel (24 g cartridge, 0-10% (0.7 M NH₃) MeOH/DCM, then 12 g cartridge, 0-7% (0.7 M NH₃) MeOH/DCM) to afford the title compound (19.6 mg, 28%) as an off-white solid. LCMS m/z=370.2 [M+H]⁺, ESI pos.

### Example 2:6-[(3R)-1-Ethyl-3-piperidyl]amino]-3-(2-hydroxy-3-bicyclo[4.2.0]octa-1,3,5-trienyl)-4-methyl-1,2,4-triazin-5-one

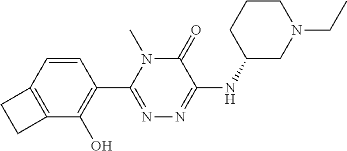 [SMILES: CCN1CCC[C@@H](Nc2nnc(-c3ccc4c(c3O)CC4)n(C)c2=O)C1]

A mixture of 3-bromobicyclo[4.2.0]octa-1 (6),2,4-trien-2-ol Intermediate 3 (100 mg, 0.50 mmol, 1.0 eq), bis(pinacolato)diboron (640.0 mg, 2.52 mmol, 5.0 eq), Pd (dppf) Cl₂·DCM Complex (50.0 mg, 0.06 mmol, 0.12 eq; CAS #95464 May 4), and potassium acetate (150.0 mg, 1.53 mmol, 3.0 eq) in 1,4-dioxane (5 mL) was degassed with N₂ for 5 minutes and then was heated to 90° C. and stirred for 6 h. The reaction was allowed to cool to rt, then K₃PO₄ (2 mL, 10% aq) was added and the reaction was stirred for 30 minutes. The reaction mixture was diluted with water (30 mL) and extracted with DCM (30 mL). The aqueous layer was acidified to ˜pH 3-4 by dropwise addition of HCl (1 M aq) and then was extracted again with DCM (5×30 mL). The combined organic extracts were dried with MgSO₄ and concentrated in vacuo. The resulting residue was dissolved in 1,4-dioxane (4 mL) and 3-chloro-6-[(3R)-1-ethyl-3-piperidyl]amino]-4-methyl-1,2,4-triazin-5-one Intermediate 1 (140.0 mg, 0.36 mmol, 0.72 eq) Xphos Pd G3 (22.0 mg, 0.03 mmol, 0.05 eq), potassium carbonate (210.0 mg, 1.52 mmol, 3.02 eq), and water (1 mL) were added. The resulting mixture was degassed with N₂ for 5 minutes, then was heated to 90° C. for 4 h. The reaction was allowed to cool to rt, was concentrated in vacuo and purified by column chromatography on silica gel (0-10% (0.7M NH₃) MeOH/DCM) to give 58 mg of the desired compound at ˜93% purity. The material was purified again by reverse phase chromatography (0.1% ammonium hydroxide, 5-35% MeCN/water) to give the title compound (33.8 mg, 18% yield) as a white solid. LCMS: m/z=356.2 [M+H]⁺, ESI pos.

### Example 3:6-[[(3R)-1-Ethyl-3-piperidyl]-methyl-amino]-3-(4-hydroxyindan-5-yl)-4-methyl-1,2,4-triazin-5-one

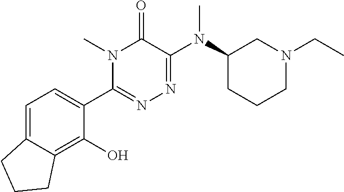 [SMILES: CCN1CCC[C@@H](N(C)c2nnc(-c3ccc4c(c3O)CCC4)n(C)c2=O)C1]

### Step A: 3-(4-Benzyloxyindan-5-yl)-6-[[(3R)-1-ethyl-3-piperidyl]amino]-4-methyl-1,2,4-triazin-5-one

2-(4-Benzyloxyindan-5-yl)-4,4,5,5-tetramethyl-1,3,2-dioxaborolane (737.0 mg, 2.1 mmol, 1.0 eq, CAS #2878443-82-2), 3-chloro-6-[[(3R)-1-ethyl-3-piperidyl]amino]-4-methyl-1,2,4-triazin-5-one Intermediate 1, (603.57 mg, 2.0 mmol, 0.95 eq), XPhos Pd G3 (178.32 mg, 0.21 mmol, 0.1 eq) and saturated aqueous sodium carbonate (3.0 mL, 2.1 mmol, 1.0 eq) were dissolved in MeCN (15 mL) and the mixture degassed with nitrogen, then stirred at 80° C. for 20 h. The mixture was diluted with EtOAc (100 mL) and washed with brine (100 mL). The organic phase was seperated, dried using a phase separator and concentrated in vacuo. The resulting residue was purified by flash chromatography on silica gel (24 g column, 0-10% (0.7 N ammonia in MeOH)/DCM) to afford the title compound (675.0 mg, 1.47 mmol, 67% yield) as a yellow solid. LCMS: m/z=460.3 [M+H]⁺, ESI pos.

### Step B: 3-(4-Benzyloxyindan-5-yl)-6-[[(3R)-1-ethyl-3-piperidyl]-methyl-amino]-4-methyl-1,2,4-triazin-5-one

3-(4-Benzyloxyindan-5-yl)-6-[[(3R)-1-ethyl-3-piperidyl]amino]-4-methyl-1,2,4-triazin-5-one (250.0 mg, 0.54 mmol, 1.0 eq) was dissolved in NMP (2 mL) and sodium hydride (60% in mineral oil, 108.79 mg, 2.72 mmol, 5.0 eq) was added. The mixture was stirred for 15 min, then iodomethane (0.04 mL, 0.65 mmol, 1.2 eq) was added. The mixture was stirred for 18 h, then further sodium hydride (60% in mineral oil, 108.79 mg, 2.72 mmol, 5.0 eq) and iodomethane (0.04 mL, 0.65 mmol, 1.2 eq) were added and the mixture stirred for a further 2 h. The mixture was quenched with MeOH (5 mL), then water (10 mL) and diluted with EtOAc (50 mL). The organic phase was washed with brine (3×30 mL), dried using a phase separator and concentrated in vacuo. The resulting residue was purified by flash chromatography on silica gel (4 g column, 0-7% (0.7N NH₃ in MeOH)/DCM) to afford the title compound (13.0 mg, 4% yield) as a light yellow solid. LCMS: m/z=474.5 [M+H]⁺, ESI pos.

### Step B: 6-[[(3R)-1-Ethyl-3-piperidyl]-methyl-amino]-3-(4-hydroxyindan-5-yl)-4-methyl-1,2,4-triazin-5-one

3-(4-Benzyloxyindan-5-yl)-6-[[(3R)-1-ethyl-3-piperidyl]-methyl-amino]-4-methyl-1,2,4-triazin-5-one (13.0 mg, 0.02 mmol, 1.0 eq) was dissolved in EtOH (1 mL) and Pd (OH) 2 (10% wt on carbon, 7.53 mg, 0.0 mmol, 0.2 eq) was added. The mixture was stirred under H₂ (2 bar) for 24 h, then the reaction mixture was filtered through a plug of celite and the filtrate concentrated in vacuo. The resulting residue was purified by flash chromatography on silica gel (4 g column, 0-10% (0.7N NH₃ in MeOH/DCM) to afford the title compound (7.0 mg, 73% yield) as an off-white solid. LCMS: m/z=384.3 [M+H]⁺, ESI pos.

### Example A

A compound of the formula can be used in a manner known per se as the active ingredient for the production of tablets of the following composition:

| | Per tablet | |
|---|---|---|
| Active ingredient | 200 | mg |
| Microcrystalline cellulose | 155 | mg |
| Corn starch | 25 | mg |
| Talc | 25 | mg |
| Hydroxypropylmethylcellulose | 20 | mg |
| | 425 | mg |

### Example B

A compound of the formula can be used in a manner known per se as the active ingredient for the production of capsules of the following composition:

| | Per capsule | |
|---|---|---|
| Active ingredient | 100.0 | mg |
| Corn starch | 20.0 | mg |
| Lactose | 95.0 | mg |
| Talc | 4.5 | mg |
| Magnesium stearate | 0.5 | mg |
| | 220.0 | mg |

1. A compound selected from
6-[[(3R)-1-ethyl-3-piperidyl]amino]-3-(4-hydroxyindan-5-yl)-4-methyl-1,2,4-triazin-5-one;
6-[[(3R)-1-ethyl-3-piperidyl]amino]-3-(2-hydroxy-3-bicyclo[4.2.0]octa-1,3,5-trienyl)-4-methyl-1,2,4-triazin-5-one; and
6-[[(3R)-1-Ethyl-3-piperidyl]-methyl-amino]-3-(4-hydroxyindan-5-yl)-4-methyl-1,2,4-triazin-5-one;
or a pharmaceutically acceptable salt thereof.

2. A compound according to claim 1, wherein the compound is 6-[(3R)-1-Ethyl-3-piperidyl]amino]-3-(4-hydroxyindan-5-yl)-4-methyl-1,2,4-triazin-5-one, or a pharmaceutically acceptable salt thereof.

3. A compound according to claim 1, wherein the compound is 6-[(3R)-1-ethyl-3-piperidyl]amino]-3-(2-hydroxy-3-bicyclo[4.2.0]octa-1,3,5-trienyl)-4-methyl-1,2,4-triazin-5-one, or a pharmaceutically acceptable salt thereof.

4. A compound according to claim 1, wherein the compound is 6-[(3R)-1-Ethyl-3-piperidyl]-methyl-amino]-3-(4-hydroxyindan-5-yl)-4-methyl-1,2,4-triazin-5-one, or a pharmaceutically acceptable salt thereof.

5-6. (canceled)

7. A pharmaceutical composition comprising a compound according to claim 1 and a therapeutically inert carrier.

8-14. (canceled)

15. A method of inhibiting NLRP3 in a subject in need thereof, which method comprises administering to the subject an effective amount of a compound according to claim 1.

16. A method for treatment of a disease, disorder or condition in a subject in need thereof, which method comprises administering to the subject an effective amount of a compound according to claim 1, wherein the disease, disorder or condition is selected from asthma and chronic obstructive pulmonary disease.

17. A method for treatment of a disease, disorder or condition in a subject in need thereof, which method comprises administering to the subject an effective amount of a compound according to claim 1, wherein the disease, disorder or condition is selected from Parkinson's disease and Alzheimer's disease.

18. A pharmaceutical composition comprising a compound according to claim 2, and a therapeutically inert carrier.

19. A method for treatment of a disease, disorder or condition in a subject in need thereof, which method comprises administering to the subject an effective amount of a compound according to claim 2, wherein the disease, disorder or condition is selected from asthma, chronic obstructive pulmonary disease, Parkinson's disease and Alzheimer's disease.

20. A pharmaceutical composition comprising a compound according to claim 3, and a therapeutically inert carrier.

21. A method for treatment of a disease, disorder or condition in a subject in need thereof, which method comprises administering to the subject an effective amount of a compound according to claim 3, wherein the disease, disorder or condition is selected from asthma, chronic obstructive pulmonary disease, Parkinson's disease and Alzheimer's disease.

22. A pharmaceutical composition comprising a compound according to claim 4, and a therapeutically inert carrier.

23. A method for treatment of a disease, disorder or condition in a subject in need thereof, which method comprises administering to the subject an effective amount of a compound according to claim 4, wherein the disease, disorder or condition is selected from asthma, chronic obstructive pulmonary disease, Parkinson's disease and Alzheimer's disease.
